(19) United States
(12) Patent Application Publication
(10) Pub. No.: US 2023/0191135 A1
(43) Pub. Date: Jun. 22, 2023

(54) Monitoring temporary pacing devices

(71) Applicant: Cardiac- Tech Limited, Oxford (GB)

(72) Inventor: Daniel Meese, Witney (GB)

(21) Appl. No.: 18/015,135

(22) PCT Filed: Jun. 29, 2021
(86) PCT No.: PCT/EP2021/067884

§ 371 Date: Jan. 9, 2023

(30) Foreign Application Priority Data
Jul. 14, 2020 (GB) 2010794.2

(51) Int. Cl. A61N 1/37; A61N 1/02; A61N 1/372

(52) U.S. Cl. CPC A61N 1/371; A61N 1/025; A61N 1/37247; A61N 1/37258

(57) ABSTRACT

Apparatus and methods for the management and safety monitoring of temporary cardiac pacing devices adapted to monitor a cardiac pacing device, the apparatus comprising; electrical connections with the heart and with the pacing device; a signal acquisition module adapted to acquire via the electrical connections cardiac signals indicative of cardiac operation, pacing impulses emitted by the pacing device, evoked signals emitted from the heart in response to the pacing impulses, and any unidentified noise signals; a processor adapted to receive from the signal acquisition module and to analyse the cardiac and evoked signals, the pacing impulses and any noise signals; a data store, and a display, wherein the processor is adapted to: i. establish a base level operation of the heart and pacing device and to store the associated quality, size and/or timing values of the cardiac and evoked signals and the pacing impulses in the date store; ii, receive instantaneous values of quality, size and/or timing values of the cardiac and evoked signals and the pacing impulses and to cause the display to show these values; iii. compare the instantaneous values against the values in the data store to establish differences therebetween; iv. analyse; a, any noise signal received, b. the instantaneous values received at step ii above, and c. any difference(s) at step iii above in terms of its/their quality, size and timing, and v, raise an alarm in the event a noise signal occurs and/or no evoked signal is received,

### FIELD OF THE INVENTION

The present invention relates to apparatus and methods for monitoring the operation of a temporary cardiac pacemaker, particularly but not exclusively in a human patient.

### BACKGROUND

Following open heart surgery, patients may experience heart rhythm disturbances which can lead to serious complications during recovery. To counter this, cardiac surgical patients are typically fitted with a temporary pacing system, or pacemaker, to regulate the heart's rhythm and ensure a near-normal cardiac output is maintained during recovery. Such systems work by “pacing”, i.e. by delivering electrical impulses (or “pacing spikes”) at predetermined energy levels to the specific locations on the heart at controlled intervals.

Post-surgical temporary pacing therapy is achieved (in most cases) by attaching a specialised pacing wire to either the epicardial (outer) surface of the heart or the endocardial (inner) surface of the heart, feeding the wire through the patient's abdomen (or out through a vein in the patient's leg or neck), and attaching the wire to an extension cable which is inserted into the temporary pacing device. Such pacing wires are known to be both unstable and subject to degeneration.

This instability, coupled with devices that lack the sophistication seen in permanent pacing, as well as device management being undertaken by the Intensive Treatment Unit (ITU) doctors and nurses who lack the expertise and experience of cardiologists and cardiac physiologists, can lead to a failure to identify acute and sudden changes in device function or patient underlying rhythm. As a result, cardiac output may be affected, resulting in higher use of inotrope (muscle contraction) medication and an extended and costly ITU stay. Other, more serious, consequences of a failure to recognise changes in pacing function can result in cardiac arrest and patient death.

Hospital audits regularly identify serious problems in the management of temporary pacing in the post-surgical ITU setting. These investigations report high incidences of failure to recognise loss of capture (the delivery of a pacing impulse that has insufficient energy to cause the heart to contract), oversensing (the pacemaker sensing a signal that is not a cardiac signal leading to inappropriate inhibition of pacing), undersensing (the pacemaker not sensing a cardiac signal and so continuing to pace when it should inhibit), poor pacing mode selection (the pacing mode instructs the pacemaker how it should function in relation to the patient's underlying cardiac rhythm) and other errors in programming, all of which can be extremely hazardous to patients.

Although patient safety is the paramount concern, questions also exist into the extent of how appropriate device optimisation (that is, ensuring that the system has the correct settings for the patient's underlying rhythm) can aid recovery. Studies have found that patients with intact and normal cardiac conduction across their atria-ventricular node (the electrical junction between atria and ventricles) had better blood pressure when the device was optimised in such a way that it allowed the ventricles to contract without the use of pacing. It has also been shown that patients with correct mode selection experience a better clinical outcome.

Pacemakers generally come in two forms. Single chamber devices where only a single lead is used to place an electrode in either the atrium or the ventricle, or dual chamber where two leads (one in the atrium and one in the ventricle) work together. A third less common type of temporary pacemaker exists that uses a third lead which is attached to the left ventricle to enable the re-synchronisation of left and right ventricles in the context of heart failure.

Generally, pacemakers work using a timer based upon the base rate (the minimum rate the device will pace at) which is recorded in pulses per minute (ppm). When a conventional pacemaker sees a signal, that signal resets the timer of the pacemaker so the device does not deliver a pacing spike. In a single chamber device (single chamber modes are called AAI or VVI depending on which chamber is being paced (i.e. atrial or ventricular)) set to pace at 60 ppm, the timing window, which works in milliseconds (ms), will be 1000 ms. Therefore, every 1000 ms, provided that there has been no intrinsic beat to reset the timer (pacemakers are designed to hold back pacing in the presence of an intrinsic beat so as not to compete with the patient's own rhythm), the pacemaker will deliver a stimulus to make the heart contract. In a dual chamber system set to the same base rate, two timers run in sequence, the Atrial-Ventricular timer (A-V timer) and the Ventricular-Atrial timer (V-A timer) to generate contractions between the top and bottom chambers, resembling a normal heartbeat. The A-V timer is usually programmed at 200 ms, but this number is adjustable. The V-A timer is set at the remaining time after the A-V timer has been subtracted from the base rate. So, in this case, 800 ms. Again, should an intrinsic signal be seen in either chamber, then the timer for that chamber will reset and the device withholds pacing for that chamber.

Bi-ventricular pacing is another feature of pacing that is important, albeit less common. The need for bi-ventricular pacing exists in patients suffering from heart failure. Heart failure is caused by both right and left ventricles beating out of time with each other. Bi-ventricular pacing is used to resynchronise ventricles back towards near normal function and thus reduce the effects of heart failure; this form of pacing is achieved using a further timer within the V-A timer known as the V-V timer.

Because a temporary pacemaker is usually fitted to a patient for a limited time only, monitoring of the operation of the temporary pacemaker and of the patient's physiology are usually carried out by an experienced person, such as a nurse or doctor; the attention such people can give any one patient is limited, but this does not significantly detract from the attention they are able to give to all of their patients because the situation is temporary. In time it is normally hoped that the patient's condition and cardiac function will settle down to a routine operation, or if not a more permanent pacemaker can be considered. Nevertheless, monitoring of a temporary pacemaker does require attention by personnel specifically trained to deal with cardiac matters (because of the gravity of cardiac problems) but also with the complex interaction between pacing devices and the heart. The need for monitoring personnel to be trained in this latter aspect means that many otherwise experienced medical personnel (i.e. doctors and nurses without specific training/experience) are not well-equipped to monitor patients with temporary pacemakers. Moreover, there are a variety of different problems which arise with temporary pacemakers that can be hazardous to the patient, these include:
Oversensing—The pacing device sees a signal that is not cardiac in nature, but responds to it by withholding pacing
Undersensing—The pacing device fails to see an intrinsic heart signal (from either atria or ventricles) and thus continues to pace when not required
Loss of Capture—The pacing device has delivered an impulse at the correct time, but the impulse is of an insufficient energy to cause the heart to contract
Mode selection—Incorrect mode selection based on the patients underlying rhythm can lead to suboptimal haemodynamics, an increased use of inotropes and a prolonged ITU stay

The above problems are particularly acute for patients whose pacemaker is only temporary, and may also occur (albeit less frequently) in patients with long term pacemakers. In addition, patients with temporary pacemakers can display problems which are common with long term pacemakers, such as arrhythmia.

### SUMMARY OF THE INVENTION

The present invention is predicated on the realisation that providing a means for monitoring temporary pacing devices which directly monitors the signals passing to and from the heart and from the temporary pacemaker can not only relieve the need for the monitoring of such devices to be overseen by specifically trained personnel, and address the problems associated with both temporary and long term pacemakers, but also simultaneously provide monitoring and diagnostic functionalities which are usually the preserve of the more experienced doctors, specialists or surgeons. This reduces the amount of attention a cardiac patient requires from such a senior medic and allows purely short term and non-critical disturbances in cardiac and/or pacing device operation to be normalised automatically but more serious disturbances to be alerted for the appropriate human intervention.

The present invention therefore provides apparatus adapted to monitor a cardiac, pacing device, particularly a temporary cardiac pacing device, which is connected to the heart via epicardial or endocardial leads, the apparatus comprising:

electrical connections with the heart and with the pacing device;

a signal acquisition module adapted to acquire via the electrical connections cardiac signals indicative of cardiac operation, pacing impulses emitted by the pacing device, evoked signals emitted from the heart in response to the pacing impulses, and any unidentified noise signals;

a processor adapted to receive from the signal acquisition module and to analyse the cardiac and evoked signals, the pacing impulses and any noise signals;

a data store, and

a display,

wherein the processor is adapted to:
i. establish a base level operation of the heart and pacing device and to store the associated quality, size and/or timing values of the cardiac and evoked signals and the pacing impulses in the data store;
ii. receive instantaneous values of quality, size and/or timing values of the cardiac and evoked signals and the pacing impulses and to cause the display to show these values;
iii. compare the instantaneous values against the values in the data store to establish differences therebetween;
iv. analyse:
   a. any noise signal received,
   b. the instantaneous values received at step ii above, and
   c. any difference(s) at step iii above in terms of its/their quality, size and timing, and
v. raise an alarm in the event a noise signal occurs and/or no cardiac or evoked signal is received.

Such an arrangement is capable of being set up by an appropriately-trained medic, nurse or allied health professional, who can establish that the base operation of the heart and the pacing device is correct and neither is operating abnormally in any observable way. The apparatus can then be allowed to monitor both cardiac function and pacemaker operation without further operator input or attention being required, unless something untoward occurs and an alarm is raised (the alarm can be both shown on the display (or User Interface) and by an audible or other visual alert, it could also be communicated to a secondary device at a remote location such as to a medic or nurse who is not in the patient's vicinity, such as by WiFi or Bluetooth to that person's mobile phone or other internet-enabled device or User Interface (UI), for example). It is also possible for the apparatus at the analysis step iv to discriminate between the types of noise signals and to give an indication of oversensing when appropriate, to assess the significance of the instantaneous values received at step ii for mode selection purposes or for sensing arrhythmia and, when no evoked signal is sensed following a pacing impulse, to determine if this is due to loss of capture, when pacing timer is not reset when an intrinsic heart signal is present to give an indication of undersensing and to assess the significance of the qualitative, size and/or timing difference(s) for mode selection purposes or for sensing arrhythmia.

The processor may be adapted to operate according to predetermined algorithms, such as those described below, to monitor a temporary pacing device for its basic operation, to monitor for oversensing, undersensing, loss of capture, far field oversensing, and arrythmia. An advantage of the present invention is that all of the algorithms can run concurrently or simultaneously, and in any combination either simultaneously or concurrently (in certain circumstances it may be desirable to monitor one, two or more functions whilst other functions may be less important (or less likely to occur in a particular patient) and so these can be monitored at a reduced frequency, so embodiments of the invention allow the associated algorithms to be run in the appropriate manner and frequency).

The processor may also be adapted to respond to operator inputs to carry out QT analysis of cardiac function, and cardiac rhythm analysis in order to suggest adjustments to the pacing device operating parameters, according to other predetermined algorithms such as those described below. The processor may be adapted to record data for real-time or offline analysis. The processor may also provide optimisation suggestions based on this analysis.

The data store may be adapted to contain causal data and values relating to the causes of cardiac arrythmia and related pacing device settings appropriate for each cause, and the processor may be adapted when requested to perform rhythm analysis to compare instantaneous values with those in the data store, identify the cause which best matches the causal data and values in the data store and to display related pacing device settings as a suggested optimisation change, so that the patient receives the most appropriate pacing impulses from the pacing device. The data store may contain pre-recorded electrograms and predetermined timings and values associated with different cardiac functions and/or malfunctions, and the pacing values associated therewith.

The apparatus may incorporate a communications module, which can not only communicate alerts to secondary devices but also allow remote configuration of the apparatus, and the raising of alerts and monitoring remotely. The user interface (UI) for this can be via a communications channel to a remote UI. Additionally or alternatively, embodiments of the invention may provide the following functionality:
Comparison of the timings of pacing impulses to the settings of the monitored device and assessment of how appropriate that timing is.
Ability to sense the evoked response generated by the pacing impulse to the epicardium/myocardium to establish that the impulse captured the heart.
Comparison of the timings of pacing impulses to those of intrinsic intracardiac electrograms (EGM) and to recognise if the monitored device has sensed or under-sensed the intrinsic heartbeat.
Use intracardiac electrograms from both atrial and ventricular pacing leads to assess and diagnose patient's underlying rhythm.
Use intracardiac electrograms to recognise changes inpatient's underlying rhythme
Establish the difference between intrinsic heart rhythm and noise.
Recognise onset of rhythms, determine if the monitored device was at fault and save the electrograms for further analysis.
Use intracardiac electrogram timings from both atrial and ventricularsignals to recommend optimal pacing parameters for the patient.

The apparatus may also be able to integrate data from additional biomedical sensors, and/or may be able to send live or recorded data to a secondary device in a processed or unprocessed state. The invention is primarily described herein in relation to the monitoring of temporary pacing devices, but it will be understood that the principles of the invention could be extended in some cases to the monitoring of long-term pacemakers.

The invention extends also to methods of monitoring the functioning of temporary pacing apparatus, as described herein.

### BRIEF DESCRIPTION OF THE DRAWINGS

The invention will now be described by way of example and with reference to the accompanying figures, in which;

FIG. 1 is a schematic illustration of a temporary pacing management and safety (TPMS) monitoring device connected to a monitored device and a heart via epicardial leads;

FIG. 2 is a block diagram of an in-line connected TPMS monitoring device;

FIG. 3 is a block diagram of a tap-off connected TPMS monitoring device;

FIG. 4 is a functional block diagram of a TPMS monitoring device;

FIG. 5 is a flow chart fora pacing function monitor (VVI/AAI mode);

FIG. 6 is a flow chart for pacing sensing analysis to detect noise leading to oversensing for single chamber mod (VVI/AAI mode) and dual chamber mode (DDD);

FIG. 7 is a flow chart for pacing undersensing

FIG. 8 is a flow chart for confirmation of capture;

FIG. 9 is a flow chart for pacemaker sensing analysis to far-field signals leading to oversensing for dual chamber mode (DDD);

FIG. 10 i a flow chart for QT interval analysis;

FIG. 11 is a flow chart for rhythm analysis and optimisation

FIG. 12 is a flow chart for detecting cardiac arrhythmia, and

FIG. 13 is an illustration of a TPMS monitoring device.

### DETAILED DESCRIPTION OF THE EMBODIMENTS

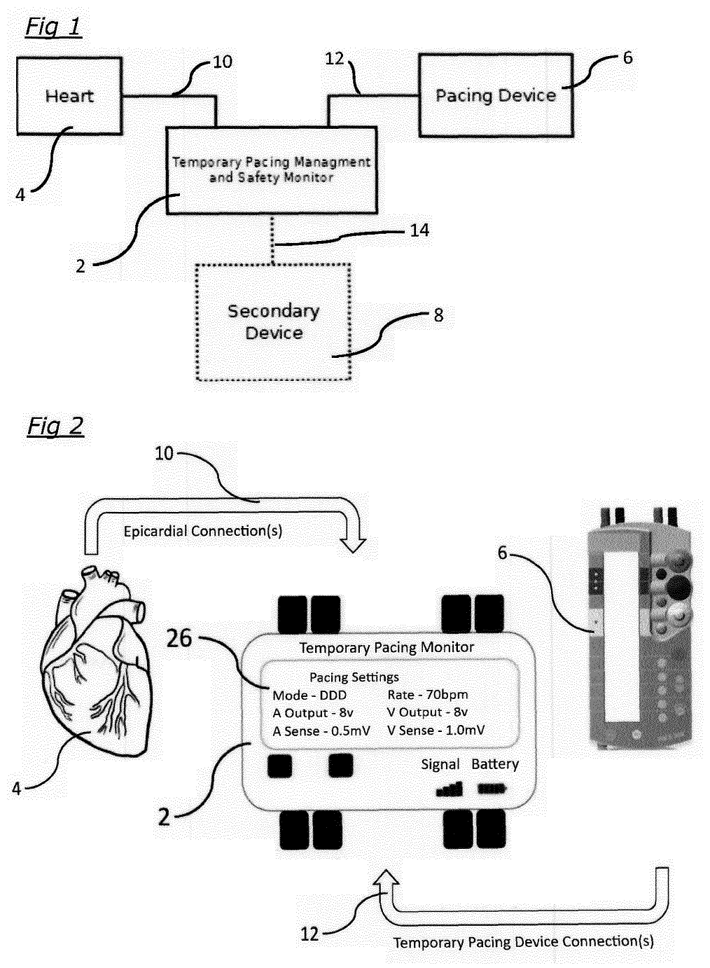

FIG. 1 shows a temporary pacing management and safety (TPMS) monitoring device 2 connected to a heart 4, a pacing device 6, or pacemaker, which is to be monitored, and a secondary device 8. The TPMS device 2 is connected electrically to the heart 4 directly via epicardial or endocardial leads 10 (wherever reference is made herein to epicardial leads it should be understood that these could alternatively be endocardial leads, or a mixture of both). The monitored pacing device 6 is external to the body of the patient and is connected to the TPMS monitoring device 2 via additional electrical leads 12. The secondary device 8, which is a smartphone or some other remote electronic device, is connected to the TPMS monitoring device 2 via a communication link 14, which may be a wireless link or a hard-wired link.

FIG. 2 shows an inline arrangement of a TPMS monitoring device, in which one or more epicardial connections 10 lead to the patient's heart 4 from the TPMS monitoring device 2, and a temporary pacing system 6 is connected to the TPMS monitoring device 2, so that the three elements are connected inline. This inline arrangement is electrically similar to the tap style (described below), but offers the additional benefit of impedance measurement, which gives a greater diagnostic capability. The TPMS monitoring device 2 has a display, or local user interface, 26 (shown more clearly in FIG. 13) indicating the pacing mode (as DDD, or dual chamber mode), the pacing rate (shown as 70 bpm—the same as 70 ppm) the atrial and ventricular output voltage (both shown as 8V) and the atrial and ventricular “sense” (i.e. the sensitivity threshold) shown as 0.5 mV and 1.0 mV respectively).

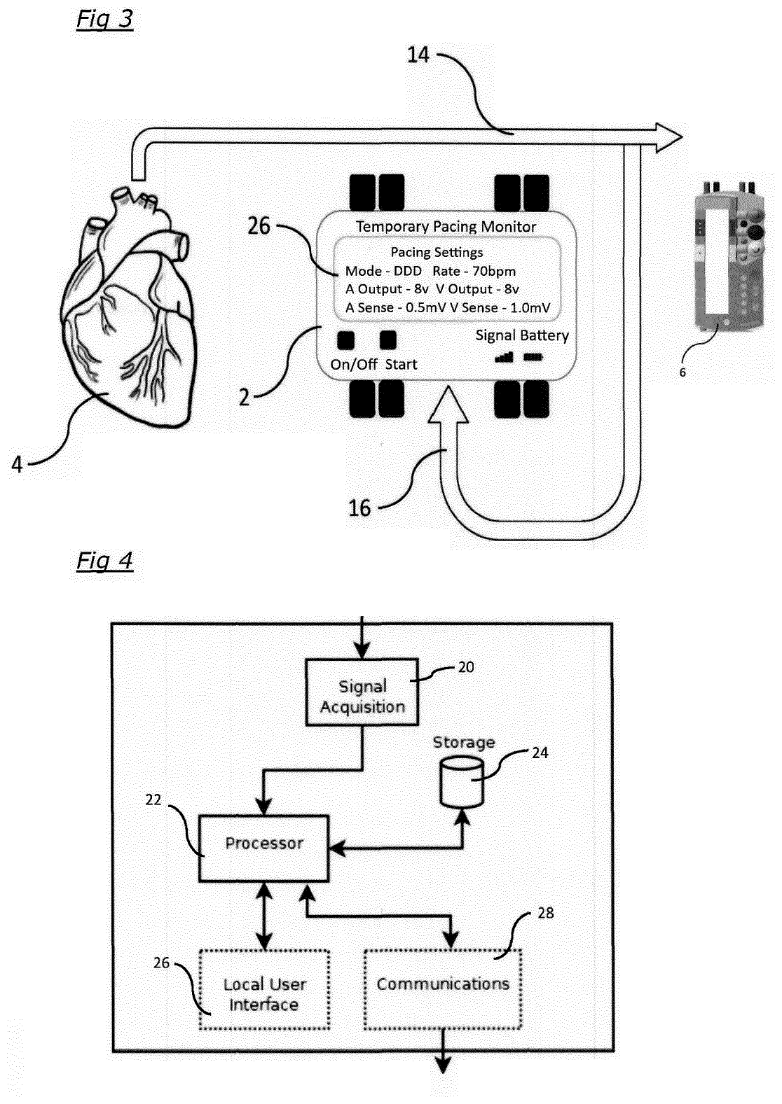

FIG. 3 shows an alternative, ‘tap-off’ style wiring arrangement for the TPMS monitoring device, in which there is a direct connection 14 (comprising one or more epicardial connections) linking the temporary pacing system 6 to the heart 4, and the TPMS Monitoring device 2 is connected by a lead 16 to the connection 14. This tap-off arrangement functions electrically in a similar fashion as does the inline arrangement, and it allows simpler wiring, but measurement of impedance or signal loading with the tap-off arrangement is not as straightforward.

FIG. 4 shows a functional diagram of a TPMS Monitoring device 2, comprising a signal acquisition module 20, a processor 22, a data storage module 24, a local user interface 26, and a communications module 28. The local user interface and the communications module are shown in shadow because either or both may be incorporated in different arrangements.

The signal acquisition module 20 provides the physical connection for the electrical signals entering the processor 22. It is designed to capture two types of signals, the epicardial signals from the heart and the pacing signals from the monitored device. The signal acquisition module 20 and processor 22 monitor all signals for noise or unexpected shape, and compare measured impedance with expected values from a database (held either in the data storage module 24 or elsewhere in the TPMS monitoring device 2) in order to ensure functional safety and to maintain signal integrity. Degenerated signals, missing signals, and/or incorrect signals are therefore detected and an appropriate response/alert is provided.

The processor 22 is responsive to signals provided by the signal acquisition module 20 and is adapted to compare the output parameters of the monitored pacing device 6 and the parameters of the signals from the epicardial leads. The signals are subjected to parametric and algorithmic analysis. A parameter change generates an audio-visual response or alarm on the TPMS device 2, and/or on a secondary device. The signals are stored in the data storage module 24. The signals are displayed on the local user interface 26, when present. The signals are transferred outside the TPMS monitoring device 2 via the communications block 28, when present. It will be appreciated that signals from the epicardial leads 10, 14 (see FIGS. 2 and 3) and also signals from the monitored device 6 are available for processing. The local user interface 26 allows the display of real-time events, alarms, and other information to the user, such as the parameters shown in FIGS. 2 and 3. It may incorporate controls (not shown) to allow parameters of the TPMS monitoring device 2 to be adjusted. The communications module 28 allows transfer of data from the TPMS monitoring device 2 to a secondary device, such as a smartphone or other remote device.

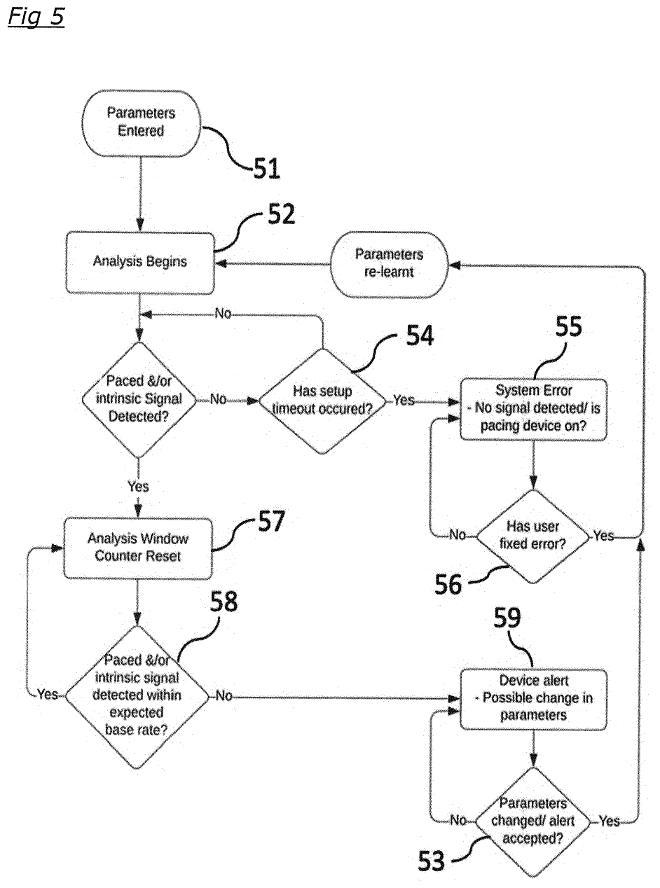

FIGS. 5 to 11 describe algorithms that the TPMS monitoring device 2 may incorporate. These algorithms may be realised procedurally, as DSP (digital signal processing) techniques or by other signal processing methods known in the art, including Fast Fourier Transforms (FFT), correlation techniques and convolution transforms.

FIG. 5 shows the operation of the TPMS monitoring device 2 during normal (non-error) conditions. After parameter setup 51 (parametric input can be manual or automatic) the device starts a control loop step 52 and waits for a signal from the pacemaker or from the patient. If no such signal arises, a timer step 54 either repeats this loop or if too much time has passed, causes an alert step 55 that can be cleared by the operator at step 56. The operator may then be invited to alter parameters at step 57 if he sees fit. If a valid signal is received, a continuous loop is set up comprising timer step 57 and comparator step 58; this loop monitors the operation of the system to ensure it is within healthy or desired parameters continuously. Should signals be received outside of acceptable time windows, then another alert step 59 is activated and the user may again be invited to change parameters at step 53, at which point the loop reverts to the main control loop at step 52.

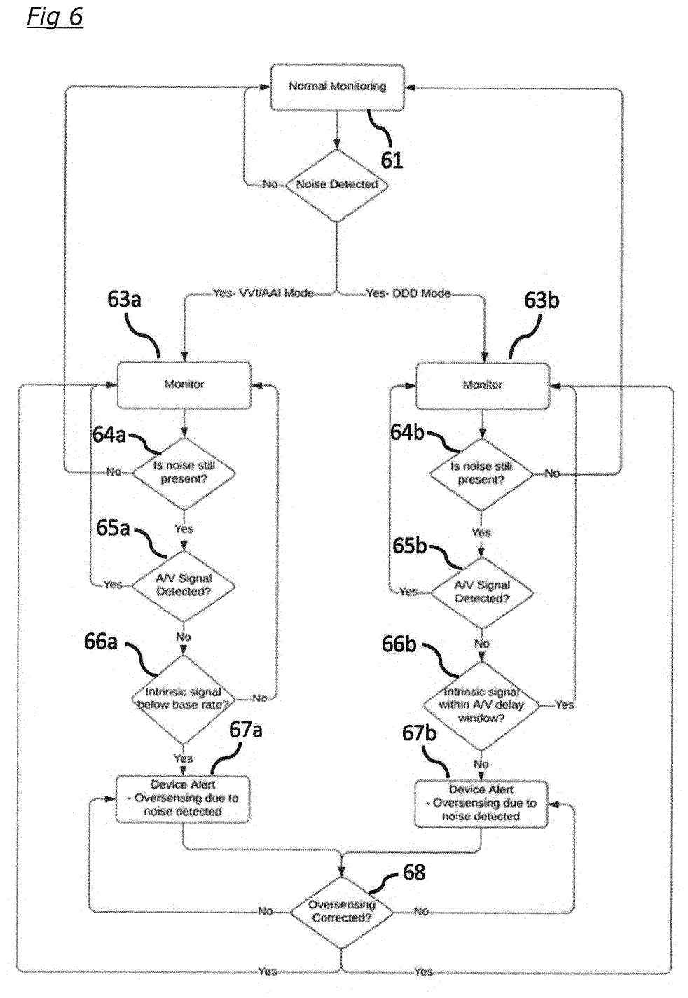

FIG. 6 shows a flowchart for detecting oversensing of noise, in both single chamber mode (i.e. VVI/AAI mode) 63a and dual chamber mode (DDD) 63b, by the monitored device 6. Once monitoring has been initiated at step 61, if no noise is detected, normal monitoring continues as described in FIG. 5 above.

If the pacemaker is operating in single chamber mode and noise is detected by the monitoring and safety device, the algorithm begins looking for paced impulses and/or intrinsic cardiac signals within the pacing timing window. This is performed at steps 64a, 65a and 66a. If no such paced impulses are seen, or intrinsic cardiac signals are detected which are slower than the device base rate (slower than the timing window), the device will issue an alarm at step 67a. If it is determined at step 64a that the noise has disappeared, the algorithm reverts to normal monitoring by entering the control loop at block 61a If noise is detected on the atrial lead in DDD mode at step 64b, the same response as the single chamber mode applies. If noise is detected on the ventricular lead by the monitoring and safety window whilst the pacemaker is in DDD mode, the algorithm looks for an intrinsic cardiac signal at the atrio-ventricular delay (AV delay) step 66b. Should no response be seen, the device will alarm at step 67b. This function is performed in a similar way to the process for single chamber mode. In either mode a decision step 68 determines if the oversensing issue has been corrected (such as by an adjustment of parameters or rearrangement of wiring) and then reverts to the previous loop at 63a for single chamber mode or at 63b for dual chamber mode.

When a conventional pacemaker sees a signal, that signal resets the timer of the pacemaker and thus the device does not deliver a pacing impulse. However, the pacemaker has no knowledge of what that signal is other than the fact that it exists. Apparatus in accordance with the invention can establish if that signal was a real cardiac signal (i.e. the device has behaved appropriately) or an extracardiac signal such as noise and thus has behaved inappropriately. At this point, the apparatus will alert the inappropriate behaviour to the nurse in charge of the patient and/or the medic responsible for the patient.

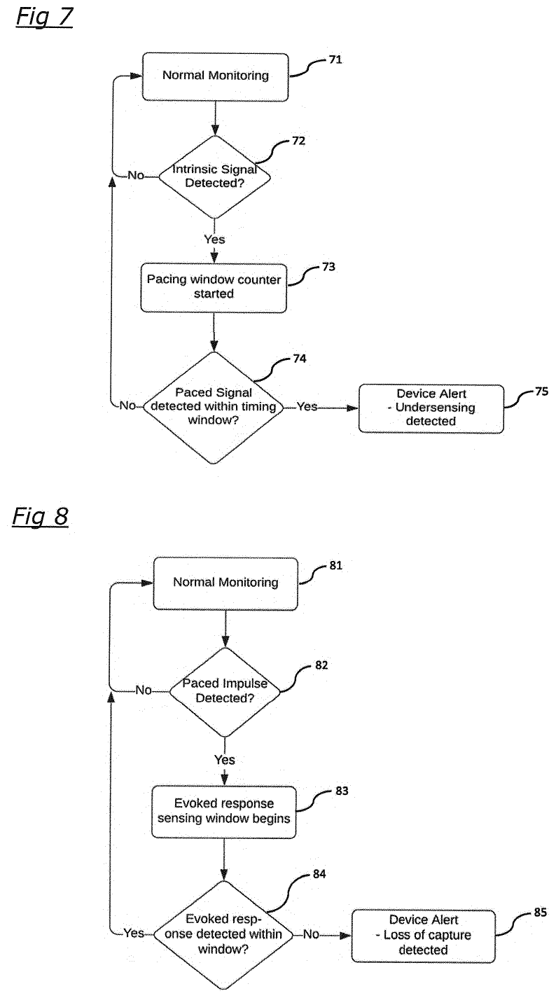

FIG. 7 shows a flowchart for detecting undersensing in both atrial and/or ventricular channels. The algorithm starts at step 71 and waits until an intrinsic (patient generated) signal occurs, at step 72. If such a signal does occur, a timer step 73 is started and step 74 looks to see if an undesirable pacing signal occurs within a certain time or not (looking if there is a signal which the pacemaker did not see, resulting in an unnecessary paced impulse). If not then the loop continues back to step 71. However if an undesirable pulse is picked up, an appropriate alert is issued at step 75. The foregoing describes an algorithm for detecting undersensing in VVI, AAI, and DDD mode. When an intrinsic heart signal is seen by the main unit from either the atrium or the ventricles, a timing window is triggered that is set to the timing set up of the pacemaker. The device then watches to see if a pacing impulse is delivered inside of this window. Should a pacing impulse be detected to be delivered, this would be considered inappropriate operation of the system and the device would alert.

FIG. 8 shows an algorithm for confirming capture by the monitored pacing device 6, that is, that the pacing device has delivered an impulse at the correct time and energy level for the heart to contract as required. The algorithm is started with an onset step 81, a decision step 82 waits for a paced impulse to be detected. Once a paced impulse has been detected the algorithm moves on to the evoked response window step 83. A decision step 84 waits for an evoked response signal to be detected within the window step 83, indicating whether or not the patient's heart has responded to the paced impulse. If an evoked response is detected within the window, the algorithm flow returns to waiting for a paced impulse at step 82. If no evoked response is detected within the window, the device alerts at step 85. The foregoing describes an algorithm for confirming capture on a monitored device 6. When a pacemaker delivers an impulse, the amount of energy used needs to be enough to trigger a heartbeat. Due to factors such as position of the epicardial lead relative to the heart, the health of the tissue the lead is attached to, and/or the quality of the contact of the lead with the heart muscle, the amount of energy required can differ for different patients. Furthermore, in temporary pacing lead parameters can change: either suddenly due to the lead moving, or slowly over time as the heart begins to endotheliorise (grow tissue) over the lead. These changes can lead to loss of capture—the amount of energy delivered is not enough to stimulate a heartbeat.

The TPMS monitoring device 2 will be able to establish capture of a paced impulse by looking for something called the evoked response. This is a signal generated by the heart muscle as the impulse causes a contraction. Once the device senses a paced impulse from the pacemaker, a window starts that looks for the evoked response. If it is seen, the device does nothing. If it fails to see the evoked response, the device alerts.

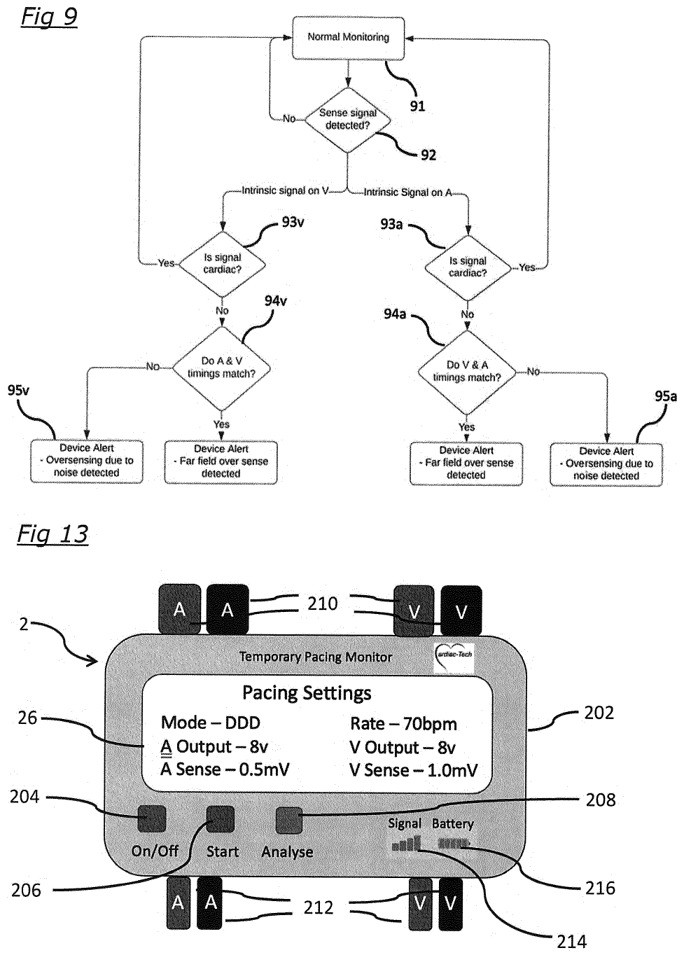

FIG. 9 shows an algorithm for detecting farfield oversensing, in single chamber VVI and AAI modes, by the monitored pacing device 6. The algorithm starts with an onset step 91 (for the continuous monitoring of pacing function, as described in connection with FIG. 5), and a decision step 92 will loop until a sensed signal is detected. When a signal is detected at step 92 a new branch is started according to whether the signal is atrial or ventricular, a further decision step 93v/93a confirms if the signal has the correct components for a near field signal (and thus from the correct chamber) or if it is deemed to be non-cardiac. If it is deemed to be near field the algorithm loops back to normal monitoring 91, if it is deemed to be non-cardiac then decision step 94v/94a assesses whether the non-cardiac signals have the same timing as the paced/sensed signals in the opposite chamber. If not, an alert is given at 95v/95a for oversensing of noise; if yes, the algorithm moves to give an alert for oversensing of farfield signals at 95v/95a. This algorithm is applied to both atrial and ventricular channels simultaneously. The foregoing describes an algorithm for detecting oversensing due to far-field signals (signals from one pacing lead detected on the opposite pacing lead) in dual chamber mode only (this kind of oversensing cannot be seen in single chamber mode). If an intrinsic signal is seen on either A or V channels, the device assesses the signal for cardiac components (near-field signals display cardiac components, far-field do not). Should it be confirmed that the signal does not have the relevant cardiac components, the safety monitor compares the A and V channel to see if the signals occur simultaneously. If they do, the device alarms for far-field over sensing. If they do not, the device alarms for oversensing of noise.

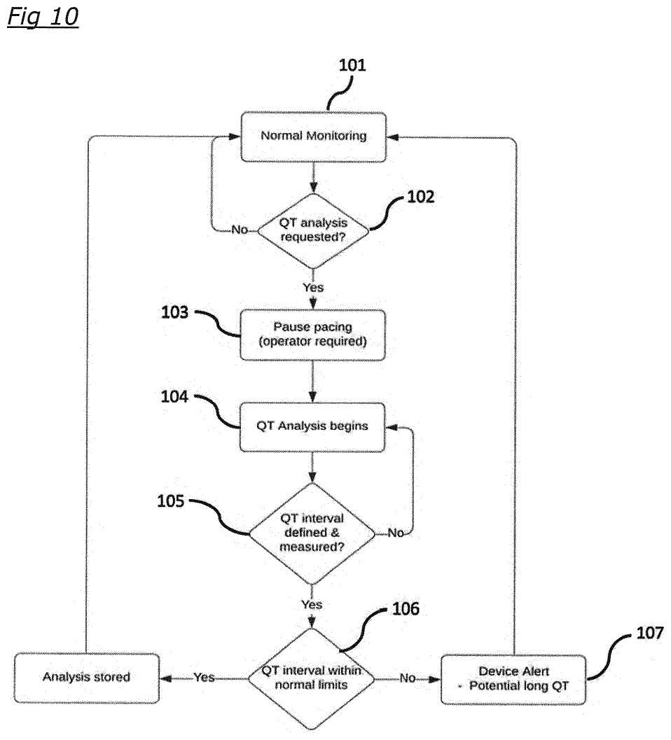

FIG. 10 shows an algorithm for QT analysis (the QT interval is the interval between the onset of the ventricular contraction (depolarisation) phase and the end of ventricular resetting (repolarisation) phase). The analysis begins with QT analysis being requested at step 102 during normal monitoring 101, and begins (after an option at 103 to pause the system) at 104 with the sampling of a number of beats to determine the current QT interval. After measurement of the QT interval 105 a decision step 106 checks if the determined QT interval is within limits. If the QT interval is within limits the analysis continues on a beat to beat basis 104 over a defined number of beats, if the QT interval is not within limits the device alerts 107. The foregoing describes a software algorithm for QT analysis. The QT interval is defined, as the total time an impulse takes to pass through the ventricles and then for the ventricular tissue to ‘reset’ itself ready for the next contraction. The total time for this to occur usually falls between 420 ms and 460 ms. Patients whose QT interval is longer than this are considered at risk of dangerous ventricular arrhythmia. The QT interval can be changed by certain medications frequently given in the post-surgical setting and so detailed analysis of the QT interval would be beneficial for medical teams when making decisions about courses of treatment.

The QT analysis algorithm works by measuring the patient's QT interval at the onset of monitoring. It does this by measuring beats (either intrinsic or paced) and looks for a trailing signal that can be defined as the T wave. It then measures the interval between the earliest indication of contraction (in a paced beat this would be the evoked response) to the last measurable point of the T wave. If the device finds it at this point to be outside of the normal intervals, it may alert. If however, the QT interval falls within normal measurements, the device continues to measure the signal on a beat to beat basis. Should prolongation of the QT interval be seen, the device may alert.

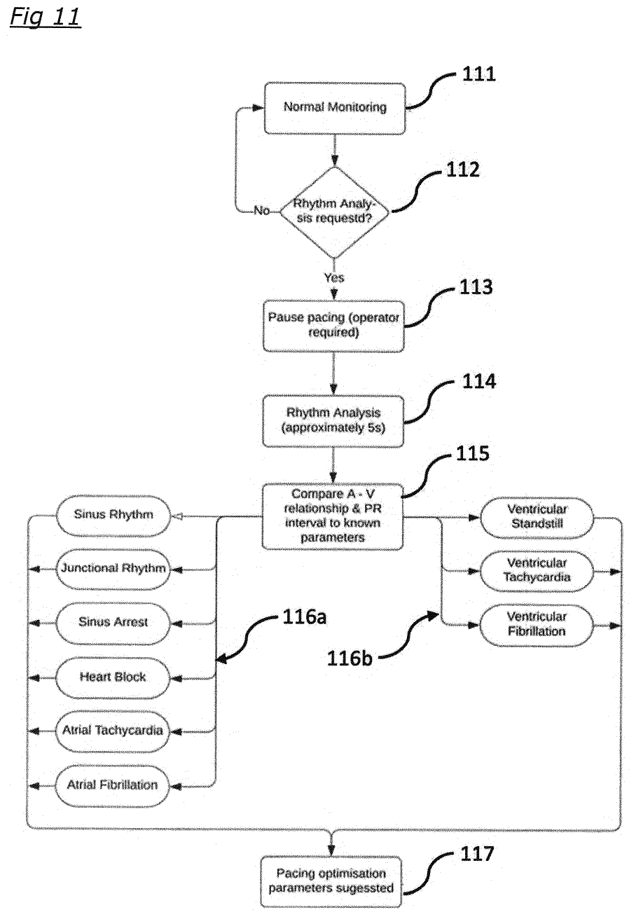

FIG. 11 shows a schematic overview of an algorithm for rhythm analysis and optimisation. During normal operation 111 a request for rhythm analysis is made at 112 and the analysis begins (after an option at 113 to pause the system) at step 114. Analysis of the rhythm is performed and, once analysis is complete, the A-V relationship between the samples is established at step 115. A database of rhythm sequences 116a, 116b is used as a comparison source. The closest match to the analysed sequence is found and the results are fed back at step 115 to the device operator as targeted suggestions 117 for modifying the settings of the monitored pacing device 6.

For bi-ventricular devices, the optimisation process includes measuring the timings between right ventricular contraction and left ventricular contraction (V to V interval), so as to calculate the required settings for appropriate re-synchronisation.

The above overview of FIG. 11 describes an algorithm for rhythm analysis to assist mode selection. Published data has shown that suboptimal pacing device programming may lead to reduced haemodynamics, increased use of inotrope medication and increased ITU stay. For example, a patient who is found to be in a rhythm called complete heart block (the impulses generated in the atrium are unable to transmit down to the ventricles leading to dissociation between the top chambers and the bottom chambers) but is only programmed to pace single chamber (ventricles only) will have a reduction in haemodynamics by as much as 30% of their cardiac output. This is due to the contractions of the atrium (which accounts for the 30% of blood moving through the heart) being out of sync with the contractions of the ventricles. By programming the pacemaker into dual chamber mode, the synchronicity of the top chambers to the bottom is restored and thus cardiac output is restored to 100% of the patient's normal volume.

For the analysis to be undertaken, a pause in pacing is required (this needs to be done on the pacemaker itself by reducing the pacing rate or pausing pacing function). To account for this and to reduce the risk of incorrect rhythm analysis, a 3 second delay in the algorithm is initiated to allow for the pause in pacing to take place. Once this has happened, a 5 second analysis window allows the device to analyse and record the relationship between signals coming from the top and bottom chambers. It then feeds this information into a rhythm database and compares and highlights sequencing that closest matches the recorded sequence before feeding back to the operator the diagnosed rhythm. Based upon its findings the temporary pacing management and safety monitoring device may also provide programming suggestions to best optimise the pacing device for the patient.

To allow for pacing optimisation, the device may use the same algorithm as seen for the rhythm detection algorithm (FIG. 11) but with an added layer. A and V relationship is still assessed in the same way as described above, however as part of the optimisation the algorithm measures timings between the top and bottom chambers (known as the PR interval) to optimise AV delay at step 115.

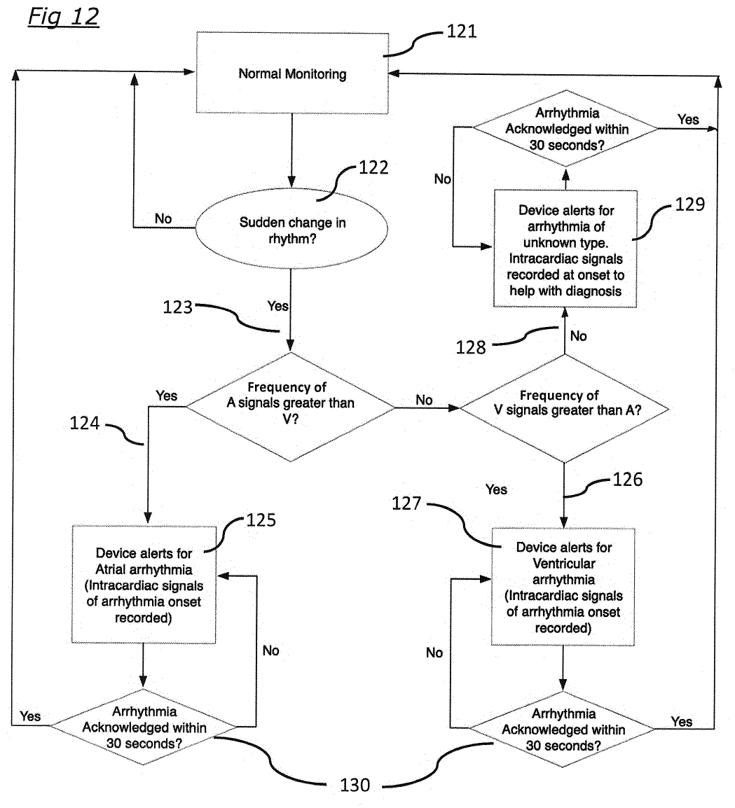

An algorithm for detecting cardiac arrhythmia is shown in FIG. 12. At the start of the algorithm, during normal operation 121 of the pacing device, the TPMS monitoring device detects 122 a sudden change in cardiac rhythm. There is then a wait to detect the frequency of A (atrial) signals compared to the frequency of V (ventricular) signals, at which point 123 the algorithm establishes which of these signals is more frequent. Should a sudden change in rhythm occur and the frequency of the A signals be greater than that of the V signals 124 a data snapshot of the arrhythmia onset is taken and an alarm is raised 125 indicating atrial arrhythmia. The operator is then required at step 130 to confirm the presence of the arrhythmia within 30 seconds. Failure to do so and a second alert is triggered. The process is repeated until confirmation of the arrhythmia's presence. If a sudden change in rhythm occurs and the frequency of the V signals is greater than the A signals 126 a data snapshot of the arrhythmia onset is again taken and an alarm is raised 127 indicating ventricular arrhythmia. Again, the operator is required at step 130 to confirm the presence of the arrhythmia within 30 seconds. If this is confirmation is not given in time a second alert is triggered. As before, the process is repeated until confirmation of the arrhythmia's presence. In the case where there is no difference in frequency between the A and V signals 128, a data snapshot of the arrhythmia onset is again taken and an alarm is raised 129 indicating the presence of an unknown arrhythmia. Again the operator is required to confirm the presence of the arrhythmia within 30 seconds. If this is confirmation is not given in time a second alert is triggered. As before, the process is repeated until confirmation of the arrhythmia's presence. If a return to A-V synchrony is detected the device may offer the user a set of suggested optimised parameters for the monitored pacing device. The device may also use electrogram template and direction analysis to identify arrhythmia originating in the ventricle. This will be used where origin of the arrhythmia is unclear.

FIG. 13 shows the TPMS monitoring device 2 in greater detail; the device receives signals from one or more I/O connections, these signals come from two sources: epicardial signals from the heart, and pacing signals coming from the monitored device. Algorithms implemented in the main unit firmware are needed to allow information to be extracted from these signals, extracted information is further used to alert the user or to provide feedback on possible adjustments to the monitored device that may be desirable. User feedback is provided to a local user via the display 3 (which may be a “touch screen”, or there may be controls, switches or the like) or to a remote user via the communications module 28, and these means may also be used to adjust variables that control behaviour of the algorithms. The display 26 provides a digital display of the pacing settings. The device has a hardened outer casing 202 made of plastic. There is an On/Off power button 204, a Start monitoring button 206 and an Analyse button 208. There are inputs for pacing leads 210 and for epicardial leads 212; these may be insulated collets that can be tightened or loosened to accept and hold or release the leads as required. There are also displays to indicate the WAP/Bluetooth signal strength 214 and the battery level 216. The device includes an alarm (not shown) which can be in the form of an audible signal and/or a visual signal, by way of a loudspeaker, buzzer, flashing light or the like.

It will of course be understood that many variations may be made to the above-described embodiment without departing from the scope of the present invention. For example, in some embodiments of the invention further algorithms may be added to encompass addition pathologies and their associated signal parameters. It will be appreciated that there may be more epicardial leads and monitored device connections than shown in the drawings. FIG. 12 is an illustration of a possible implementation of the invention. The implementation will vary depending on the embodied aspect of the invention.

It will be appreciated that it is straightforward to modify the apparatus and algorithms described above for bi-ventricular pacing (all of the pacing functionality described above can be provided by existing bi-ventricular pacing devices, which can also pace left ventricle and right ventricle independently of each other (known as V to V offset)), and that all of the monitoring features described above can be provided in bi-ventricular format simply by including a left ventricular arm, where appropriate, into each algorithm.

There may be controls provided on the TPMS monitoring device to allow differential setting of the various algorithms, to allow an operator to set which algorithms are to run simultaneously and/or at what frequency, and to set other algorithms to run concurrently and/or at a different frequency.

Where different variations or alternative arrangements are described above, it should be understood that embodiments of the invention may incorporate such variations and/or alternatives in any suitable combination.

1. Apparatus adapted to monitor a cardiac pacing device which is connected to the heart via epicardial or endocardial leads, the apparatus comprising:
electrical connections with the heart and with the pacing device;
a signal acquisition module adapted to acquire via the electrical connections cardiac signals indicative of cardiac operation, pacing impulses emitted by the pacing device, evoked signals emitted from the heart in response to the pacing impulses, and any unidentified noise signals;
a processor adapted to receive from the signal acquisition module and to analyse the cardiac and evoked signals, the pacing impulses and any noise signals;
a data store, and
a display,
wherein the processors adapted to:
i. establish a base level operation of the heart and pacing device and to store the associated quality, size and/or timing values of the cardiac and evoked signals and the pacing impulses in the data store;
ii. receive instantaneous values of quality, size and/or timing values of the cardiac and evoked signals and the pacing impulses and to cause the display to show these values;
iii. compare the instantaneous values against the values in the data store to establish differences therebetween;
iv. analyse any difference(s) at step iii above in terms of its/their quality, size and timing and to raise an alarm in the event a noise signal occurs and/or no cardiac or evoked signal is received.

2. Apparatus according to claim 1 in which the processor is adapted to follow a first predetermined algorithm when a noise signal is received to establish whether or not there has been oversensing before an alarm is raised.

3. Apparatus according to claim 1 or claim 2 in which the processor is adapted to follow a second predetermined algorithm when an inappropriately timed pacing signal is seen in relation to a cardiac signal to establish that there has been undersensing before an alarm is raised.

4. Apparatus according to claim 1, 2 or 3 in which the processor is adapted to follow a third predetermined algorithm when no cardiac signal is received to establish whether or not there has been a loss of capture before an alarm is raised.

5. Apparatus according to any preceding claim in which the processor is adapted to follow a fourth algorithm to sense arrhythmia, and to raise an alarm when arrhythmia is sensed.

6. Apparatus according to any preceding claim in which the processor is adapted to respond to an operator input request to carry out QT analysis or rhythm analysis and to follow fifth or sixth algorithms to perform the requested analysis.

7. Apparatus according to claim 6 wherein the data store is adapted to contain causal data and values relating to the causes of cardiac arrythmia and related pacing device settings appropriate for each cause, the processor being adapted when requested to perform rhythm analysis to compare instantaneous values with those in the data store, identify the cause which best matches the causal data and values in the data store and to display related pacing device settings.

8. Apparatus according to any preceding claim further comprising a visual and or audible alarm to alert an operator/medic/nurse that the functioning of the heart and/or the pacing device is incorrect.

9. Apparatus according to any preceding claim further comprising a communications module adapted to communicate to/from a user interface which is remote from the apparatus.

10. Apparatus according to any preceding claim in which the heart, the apparatus and the pacing device are connected in an inline arrangement by the electrical connections.

11. Apparatus according to any preceding claim in which the electrical connections to the heart are placed so as to provide pacing signals to one of or to both of the atrial and ventricular chambers.

12. Apparatus according to any preceding claim in which the processor is adapted to integrate and utilise data from additional biomedical sensors.

13. A method of use of the apparatus according to any one of claims 1 to 12 in which the apparatus is monitored by an operator during an initial, set up stage to ensure that the base levels established by the processor are correct and do not correspond to any inappropriate functioning of the heart or the pacing device.
